(19) United States
(12) Patent Application Publication
(10) Pub. No.: US 2010/0217275 A1
(43) Pub. Date: Aug. 26, 2010

(54) DEVICE FOR INDUCING VIBRATIONS IN A GUIDEWIRE

(76) Inventors: Ran Carmeli, Rinatya (IL); Jonathan Einav, Raanana (IL); Itai Yonat, Tel Aviv (IL)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/516,431

(22) PCT Filed: Nov. 21, 2007
(86) PCT No.: PCT/IL07/01435

§ 371 Date: Mar. 10, 2010

(30) Foreign Application Priority Data
Nov. 27, 2006 (IL) 179618

(51) Int. Cl. A61B 17/22

(52) U.S. Cl. 606/128

(57) ABSTRACT

The invention relates to the field of minimal invasive catheterization, in particular to an apparatus for opening and/or removing obstructions occluding body internal passages by means of an active guidewire comprising a coil to which an alternating voltage can be applied. In that way the guidewire can vibrate if an external magnetic field is applied.

### FIELD OF THE INVENTION

The present invention relates to the field of minimal invasive catheterization. In particular, the invention relates to an apparatus for opening and/or removing obstructions occluding body internal passages by means of an active guidewire. More particularly, the invention relates to an active oscillating guidewire, capable of passing through an occluded vessel.

### BACKGROUND OF THE INVENTION

Many vasocclusive events, such as heart attacks and strokes, are caused by plaque build-ups in arteries. As one specific example, atherosclerotic plaque is known to build-up in the walls of arteries in the human body. Such plaque build-up restricts circulation and often causes problems, for example cardiovascular problems, especially when the build-up occurs in coronary arteries.

One common method for opening partially occluded body internal passages is to guide a medical device to the diseased site, where it is used to carry out the needed treatment. A guidewire is usually used for advancing a catheter device thereover via body internal passages towards the treatment site. Typically, the distal tip of the guidewire is introduced into the body of the treated subject via an incision and advanced therethrough towards the treatment site, thereby forming a path leading to the occluded site through said body internal passages. A catheter, or any other suitable treatment devices (e.g., balloon catheter, stent, rotational atherectomy device, laser device etc), may be then threaded over the guidewire and advanced through said internal passages using the guidewire as a rail.

Total or near-total occlusions in body internal passages can, partially or entirely, block the passage therethrough. For example, in patients who suffer from coronary chronic total occlusion (CTO), the successful performance of a Percutaneous Transluminal Coronary Angioplasty (PTCA) is a technical challenge. The factor that is most determinative of whether the practitioner can successfully perform PTCA on patients suffering from coronary CTO is his ability (or inability) to advance a suitable guidewire from a position proximal of the lesion to a position distal of the lesion while remaining inside the true vessel lumen (without performing perforation of the artery wall).

In some instances, such when the occlusive matter is soft or where the body internal passage is partially occluded, the guidewire can easily be pushed through the occlusive matter itself, thereby allowing the guidewire to remain within the body internal passage. However, in other cases, such as when the body internal passage is totally occluded by hard plaque (e.g., calcified atherosclerotic plaque), the guidewire cannot cross the occlusion and may deviate to the side and penetrate through layers of the passage walls (e.g., the intima—inner layer of a vessel wall), thereby creating a neo-lumen therethrough (e.g., through the sub-intimal space—within the wall of the artery between the intima and media, or adventitia i.e. a dissection), or even completely exit said internal passage i.e. a perforate the passage wall.

Several techniques are known for passing through an occluded internal passage, such as laser catheters (U.S. Pat. No. 6,673,064), ultra sonic catheters (U.S. Pat. No. 6,702,748), and tissue displacement or hinged expansion devices (U.S. Pat. No. 6,800,085). In all of those techniques the occlusion is opened by means of a catheter device equipped with operative means for opening of occlusions. However, the prior art devices suffer from lack of flexibility and maneuverability due to the bulky structure of their catheter devices. Consequently, the treatment procedures that utilize these prior art devices are substantially different from conventional catheterization procedure workflow as commonly practiced in regular cases (non CTO cases).

Other known procedures, such as described in U.S. Pat. No. 6,852,109, propose a method for forming a passage through the CTO by a guidewire having active Radio Frequency (RF) ablation tip, with Optical Coherence Reflectometry (OCR) capability for sensing the position of the tip. However this known type of guidewire is a special guidewire comprising a mechanism for transferring RF energy and a following catheter with fiber optics for the OCR capability. These restraints are relatively rigid and therefore diminish the flexibility of the device, which is an important and necessary feature for carrying out in vivo navigation. Thus, also this prior art device suffers from lack of flexibility, and the need to deviate from the conventional practice workflow of the practitioner. Furthermore, this apparatus comprising a guidewire and a catheter works well only if the physician tracks the guidewire with the catheter—as the OCR requires both the guidewire and the catheter.

A prior art solution for determining whether an organic tissue is healthy or not is using IVUS (intravascular ultrasound), for example as described in U.S. Pat. Nos. 6,685,644 and 6,685,643, however lack of data due to poor transmission in this known method and calcified build-ups that cause “acoustic shadowing” yield poor results in determining tissue type and true lumen detection.

In still another solution used such as described in U.S. Pat. No. 5,908,395, a hand held vibrator is attached to the proximal side of a guide wire, or a catheter through which the guidewire is threaded. The guidewire then conveys the proximal vibrations to its distal end, subject to the specific passage of the guidewire through the arteries. If indeed these vibrations reach the distal end of the guidewire they may be efficient in penetrating and recanalization of CTOs). However, there is a problem in that the vibrations may be absorbed before reaching the distal end. In addition, using an external hand held vibrator interferes with the standard operation of the guidewire, and limits the operator from conveniently controlling the guidewire by manually holding its proximal end, again causing the need to deviate from the conventional practice workflow of the practitioner.

In still another solution used as described in copending application PCT/2006/000541, a device comprises a magnetic guidewire housed in a coiled catheter. The guidewire is vibrated by feeding electrical current via the coils of the catheter, thus providing magnetic excitation of the guidewire tip. However, because of the magnetic guidewire structure and dimensions this solution may not be optimized to match the physician procedure. The magnets bids added to the guidewire may increase the diameter thereof, and may thus prevent the use of some devices that are threaded onto regular guidewires.

More particularly, these over-the-wire devices sometimes have a lumen that is only marginally larger in diameter than the diameter of the guidewire. By adding magnets to the guidewire, these over-the-wire devices may not suited to be threaded onto the magnetic guidewire, as the diameter of the magnets may be bigger than the inner diameter of the lumen of the catheter.

In still another solution used such as described in WO 00/00252, a catheter or guidewire that is made from ferromagnetic means and is positioned such that a predetermined portion of the device lies adjacent to the target site. A magnetic field source that changes over time in magnitude and/or direction, of sufficient strength is disposed outside the patient's body in sufficient proximity to the intrabody device to induce motion in the device through the oscillating magnetic field that it emits. However this method significantly deviates from the physician regular workflow and requires the change of the catheterization lab in order to facilitate the magnet field exterior device, furthermore, it is almost impossible to guarantee that the magnetic member that lies within the body cavity will be exactly adjusted to the magnetic source.

In still another solution such as described in WO 94/12234, a flexible elongate device having a distal extremity with a vibratory impact tip embedded with a coil spring piston like mechanism for catheters and guide wires. However, because of the fact the guidewire or catheter are embedded with a spring coil vibrational mechanism, the mechanical properties of the guidewire and the catheter significantly change and the ability to pass over the wire devices is reduced, thus deviating from the standard workflow of the procedure.

Several uses of magnetic coupling of guidewires are known, such as in U.S. Pat. No. 5,813,996, however this known coupling is static and is used as a guide wire extension system including a guide wire and an extension wire and means for magnetically coupling the guide wire to the extension wire, and not as a means for magnetic vibration for gateway passage opening.

There thus exists a need for devices and techniques for treating occluded body internal passages, for characterizing the tissue/substance the treatment device is in contact with, for determining its location within and about the body internal passage, and for safely opening occlusions therein without damaging the occluded internal passage, while keeping the same work flow (clinical procedure), and enabling the physician to use exactly the same over-the-wire devices.

Further more, guidewires are a great technical and clinical challenge. The structure of the guidewire, and generally speaking composition of materials and dimensions of the different segments of the guidewire set the guidewire's characteristics. More specifically, most guidewires are constructed such that their distal portions (typically the distal 100-300 mm of the guidewire) are made of a specially shaped and tapered core, wrapped with a special spring-like coil. This coil, together with the inner shaped core of the guidewire influences dramatically the behavior and characteristics of the guidewire.

Therefore, it is desirable to be able to make use of the existing structure of conventional guidewires, and thus maintaining the critical mechanical characteristics of the guidewire, while adding the capability to generate an alternating magnetic field.

This alternating magnetic field can then serve to generate alternating magnetic forces that oscillate the tip of the guidewire providing it with active drilling capabilities to open occlusions.

It is an object of the present invention to provide a method and device for opening occluded body internal passages and or body organs, by providing additional means and implementing the coils already embedded into a guidewire in such a way so as to enable electrical generation of alternating magnetic fields.

### SUMMARY OF THE INVENTION

### The Apparatus

The present invention is directed to an active guidewire housed in a magnetic catheter based device, to a method for opening obstructed body internal passages and for sensing and characterizing tissues and substances being in contact with the device of the invention.

In general, the device of the present invention comprises a coiled guide wire, capable of inducing magnetic force(s) therein while threaded through a catheter with fixed magnets attached to it. That magnetic force can be an alternating magnetic force, creating vibrations in the tip of the guidewire, wherein said vibrations of the guidewire are utilized for opening a passage through an occlusion.

An alternative apparatus comprises coiled guide wire threaded into a human vessel, which is positioned in a strong magnetic gradient flux. This magnetic gradient flux is generated by in-vitro magnetic apparatuses', such as strong fix magnets, or strong electro-magnets.

More particularly, the present invention relates to a device and method for in vivo drilling in living tissue and/or finding the weak path in the said living tissue, in body internal passages and body organs, which may be utilized for opening a passage suitable for passing a treatment device (e.g., a stent, balloon) through an occluded body internal passage (e.g., blood vessel), such as in cases of CTO. The in vivo drilling is performed by means of a drilling guidewire with an embedded electro-magnet, and more specifically a coil based electro-magnet section that aside from drilling through the occlusion enables the operator to sense the tissue/substance being in contact with the drilling part of the drilling guidewire. The sensing of the tissue/substance contacting the drilling guidewire may be advantageously used to provide an indication as to whether the drilling guidewire properly operates in the occluded internal passage or whether it deviates therefrom and injures the passage wall. The path drilled and or opened and or re-canalized through the occluding matter enables the passage of an either the special guidewire or another conventional guidewire through the occluded passage and thereby allows carrying out the conventional treatments of “over-the-wire” applicable in such cases, such as, for example, by means of balloon catheters and stents.

The drilling according to this invention is based on an electro-magnetic force generated by the combination of feeding current through a guidewire according to the invention, threaded in a catheter with embedded magnets.

The drilling itself is carried out by means of rapid vibrations e.g., at a frequency of about several dozens Hz, preferably in the region of 1-600 Hz, low amplitude vibrations e.g., with an amplitude of about a fraction of a mm, preferably in the region of 0.01 to 1 mm, that are directed in vivo to the occluding matter. The in vivo drilling device of the invention is designed to transfer the drilling energy with high efficiency into hard/calcified tissue, while keeping the efficiency low when drilling into relatively soft tissues. The drilling vibrations are preferably limited to low amplitudes, resulting in a drilling scheme that transfers energy very efficiently into hard/calcified occlusions. At the same time, such drilling cannot transfer energy efficiently into soft tissues, such as the artery wall, thus lowering the risk of perforation or dissection thereof.

The present invention also provides a method for opening occluded body passageways by means of a drilling guidewire, which comprises a conventional guidewire that is slightly modified and which is operated (upon physician selection) in an active mode (electrically powered) as a driller held by the catheter surrounding the guidewire near its distal end. In this manner the physician can advance the guidewire as far as possible towards the occlusion while the device is in a passive mode, and upon reaching an occluded section the physician switches the device into an active mode, thereby initiating an active magnetic drilling process to allow further advancing the of the guidewire all the way to the distal end of the occlusion, and thereafter to treat the occluded passageways using conventional procedures, such as by means of balloon catheters and/or stents.

### The Guide Wire

A preferred guidewire according to the invention is based on a conventional configuration guidewire with the added capability of driving an electrical current through the coils that are a part of the guidewire.

In another embodiment of the invention, the coil already embedded in a conventional guidewire is electrically connected to an external current driving unit, thus creating an electrical flux in the vicinity of said coil. When the coil is then inserted into a magnetic field gradient, an induced magnetic force is developed which acts on the coil. This magnetic field gradient may be generated either by in-vivo means, or by in-vitro (external to the human body) means.

In yet another embodiment of this invention, the coils that are electrically connected to an external current driving unit are positioned at various places along the guidewire.

In a preferred embodiment of the invention, the coil is positioned at the front portion of the guidewire (i.e. in the region of its distal end???). The exact position of the coil may vary from one embodiment to another.

In a further embodiment of the invention, the coil distal end is positioned approximately 50 mm before the distal tip of the guidewire.

In yet another embodiment of this invention, the coil distal end is positioned just at the tip of said guidewire.

In yet alternative embodiments of this invention, the coil distal end may be positioned along the guidewire in any is at a distance, in the range of 10-300 mm away from the distal tip of the guidewire.

In yet another preferred embodiment of the invention, the coil may be divided into several gapped segments of coils, implemented in an optimized electro-magnet configuration to achieve a significant magnetic field flux along the guidewire.

In yet another preferred embodiment of the invention, the number of divided coil segments is typically in the range of 3 to 30.

All such segments can be connected in serial to each other, or in another electrical connection, which allows achieving the desired result.

In yet another preferred embodiment of the invention, the coils are connected in serial to each other in such a manner that the current direction changes form one coil to its neighboring coil. In short, assuming there are e.g. 7 coils, then all coils are connected in serial to each other, in a way that the current fed into coils 1, 3, 5 and 7 flows in “clockwise” direction, while the current fed into coils 2, 4 and 6 flows in “counterclockwise” direction.

In yet another preferred embodiment of this invention, the gap between adjacent coils will be of the order of 1-5 mm, while the length of each coil section may preferably be in the range of 0.5-10 mm.

In yet another preferred embodiment of this invention, the gaps between the coils section may be one of the following:
Left empty.
Filled with another passive coil, that is not connected to an electrical driving unit.
Filled with other material, preferably a bio-compatible type of material.
Filled with a thermal conductive material, to sink the heat dissipation generated by the coil into the metal inner member of the GW.

In yet another preferred embodiment of the invention, the coils are made of wire with diameter that is in the range of 25 to 100 microns (10 to 40 mils).

The coils are made of silver, copper, platinum, or any other material that is compatible for inserting into living organs of a human being.

In yet another preferred embodiment of the invention, the coil may be of a mechanical structure similar or identical to the mechanical structure already implemented in the guidewire, hence suitable for use in catheterization and/or treatment of live human beings.

In yet another preferred embodiment of the invention, the external diameter of the coil(s) is identical to the external diameter of the guidewire section, in which the coil(s) are embedded, typically in the range of 14 to 18 mils.

In yet another preferred embodiment of this invention, the coils are coated with isolating materials. Preferably these materials will provide clinical benefits, such as hydrophilic coating.

In yet another preferred embodiment of this invention, the coating may be of bio-compatible type, such as Teflon based coating.

In yet another preferred embodiment of this invention, the coils are connected to an external current driving unit, by means of two leads that are pulled along the guidewire up to its proximal side end. Optionally these leads can be threaded alongside the guidewire, wrapped in an isolating shrinkable sleeve, or through a hollow core of the guidewire. Alternatively, either of the leads can be a part of the core member of the proximal portion of the guidewire. It should also mentioned, that more than a single lead can be a part of the core member of the proximal portion of the guidewire, assuming it is designed in a way to be comprised of several electrically isolated cores aligned with each other. In this embodiment the leads are optionally terminated with a connector, preferably miniature connector, or as bare wires, directly connected to the external driving unit.

The present invention also provides a guidewire device, capable of electrically controlling its distal tip pushing force, according to the electrical current fed into the coils of the guidewire:
An oscillating current will result in an oscillating magnetic force, the magnitude thereof force depending on the amplitude of the current fed.
A DC current will result in a constant magnetic force, which adds to the inherent mechanical force of the tip of the guidewire, hence changing its stiffness according to the current level.

### The Catheter

The in vivo drilling of the guidewire according to the invention is achieved when threading the guidewire according to the invention into a magnetic catheter, and then feeding electrical current into the coils of the guidewire.

The catheter according to this invention may be any type of hollow tube.

Preferably, the hollow tube may be made for clinical use in general, and more particularly for use in coronary catheterization procedures.

In yet another preferred embodiment of the invention, the catheter is a conventional catheter provided with magnetic bids along it.

In yet another preferred embodiment of the invention, the magnetic bids are hollow bids, attached to the inner wall, the outer wall, or embedded in the wall of the catheter.

In yet another preferred embodiment of the invention, the magnetic bids are made of rare earth magnets, such as NdFb48. The preferred size of the said magnetic bids is preferably designed to match the design of the coils of the guidewire.

In an optional embodiment of the catheter according to the invention, the number of magnetic bids along a specific length of catheter equals the number of coil segments along same length of the guidewire.

Alternatively, the number of coils can differ from the number of magnetic bids. In this case the maximum magnetic force generated by the apparatus shall be maintained as long as coils are surrounded by magnetic bids.

In a preferred embodiment of the invention, each coil segment of the guidewire is surrounded by two magnetic bids attached to a catheter, in a manner that the magnetic field generated by the coil is positioned in a high gradient of the magnetic field generated by the two bids, hence producing a magnetic force.

In yet another preferred embodiment of the invention, the magnetic bid may be a hollow shaped cylinder, with outer diameter of 2.5 F (0.9 mm) and an inner hole diameter of 1.9 F (0.65 mm) with a length of 3 mm.

The frequency of the vibrations may be changed in close loop in order to manually or automatically reach the resonance frequency or anti-resonance of the whole apparatus, thus gaining continuous optimization of the drilling energy.

In yet another embodiment of the catheter according to the invention, the rare earth magnets of the catheter can act also as radio opaque markers, used for identification (automatically and/or manually) of the relative position between the guidewire and the catheter, and hence provide means to selectively drive coils that are overlapping the guidewire magnets. Such selective driving provides the following main advantages:
1. The power loss on the coils is lower relative to the power loss when activating all the coils (as only some of the coils are driven).
2. The working range of the magnetic force may be increased, relative to the working range when powering all of the coils.
3. The magnetic force level may be efficiently developed, as only coils that contribute to the magnetic power are activated, while other coils are selectively not powered.

### Sensing

The electrical power (current and voltage vs. frequency) fed into the vibrating elements, can be analyzed to measure the overall apparatus magnetic impedance, and thereby provide an indication about the type of tissue/substance drilled, as described in Israel copending patent application No. 168569 and in copending application PCT/2006/000541.

### BRIEF DESCRIPTION OF THE DRAWINGS

FIG. 1 shows the general structure a prior art guidewire,

FIG. 2 shows a general structure of a prior art catheter,

FIGS. 3A and 3B show a general typically usage of the prior art guidewire, when threaded for clinical treatment through a prior art catheter,

FIGS. 4A, 4B, 4C and 4D show a preferred guidewire embodiment according to the invention,

FIG. 5A shows a close up cross-sectional look of the coils according to the invention, and FIG. 5B the magnetic flux developed thereby,

FIGS. 6A and 6B show other configurations of the guidewire,

FIGS. 7A and 7B show the catheter according to the invention with magnetic bids,

FIGS. 7C and 7D show a typical magnetic field developed by the magnetic bids,

FIGS. 8A and 8B show a use of the guidewire when threaded via the catheter, to enable the desirable active drilling of the apparatus,

FIG. 9 shows another drilling apparatus based on the guidewire and the catheter,

FIG. 10 shows the leads connecting the guidewire to the external current driver, and

FIG. 11 shows a schematic block diagram of an external driver unit.

### DETAILED DESCRIPTION OF PREFERRED EMBODIMENTS

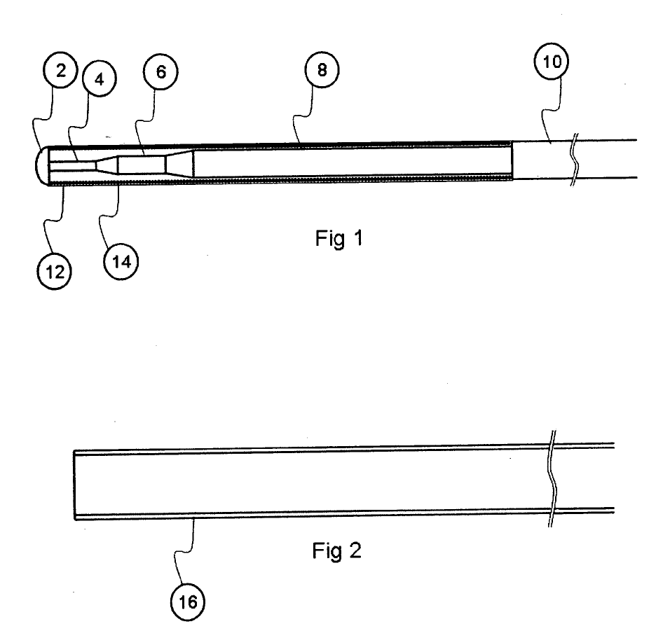

FIG. 1 shows a typical prior art guidewire. Generally speaking the guidewire is divided into 2 main zones, a working zone 8, that are inserted into a vessel in the human body during a typical procedure carried out by a practitioner and the rest of the guidewire 10, which in most cases does not touch the vessel walls, as it is typically housed in a catheter. The overall length of typical guidewires varies in the range of about 160 to 300 cm. The working zone 8, is divided into several segments. The front tip 2 of the guidewire, is the first part of the guidewire that touches the organ, and must be designed in a way so as to not to harm the organ or vessel wall. A first core member 4, follows the distal tip of the guidewire, surrounded by a spring type envelope 12. The section that contains the core member 4, typically sets the flexible zone of the guidewire enabling it to propagate safely and conveniently through the vessel. A thicker second core member 6, follows the first core member 4, forming a less flexible zone also called “stent zone”. Second core member 6 is connected to a third core member 8, which is thicker and stronger and is used to enable the pushing of the guidewire along the vessel. Some guidewires may include different numbers of zones than described in this figure, e.g. to fit special clinical needs, yet this description gives a comprehensive structure of a typical guidewire. All core members are sometimes surrounded by spring type coils 12. The spring type coils 12 cover is may in some cases be coated with special coatings, such as hydrophilic coating 14.

FIG. 2 shows a typical structure of a prior art catheter 16. The catheter 16 in a typical case forms a hollow plastic tube, capable of being inserted into human arteries. This hollow structure enables the transmission of materials, e.g. liquids, and devices, e.g. the guidewire, through its inner lumen.

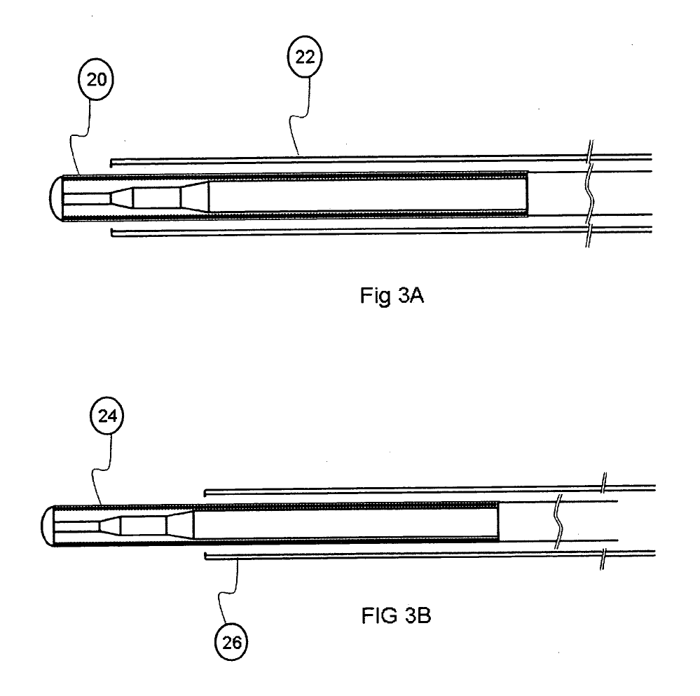

FIGS. 3A and 3B show the typical relation between a guidewire, as shown in FIG. 1 and a catheter as shown in FIG. 2. The guidewire 20 is threaded through a catheter 22, and then pushed towards the obstructed vessel to enable the treatment. It is typically capable of being to push back and forth guidewire 20 relative to catheter 22. FIG. 3B illustrates that sliding capability, where the guidewire 24 emerges to a significantly greater extent from catheter 26 than is shown in FIG. 3A.

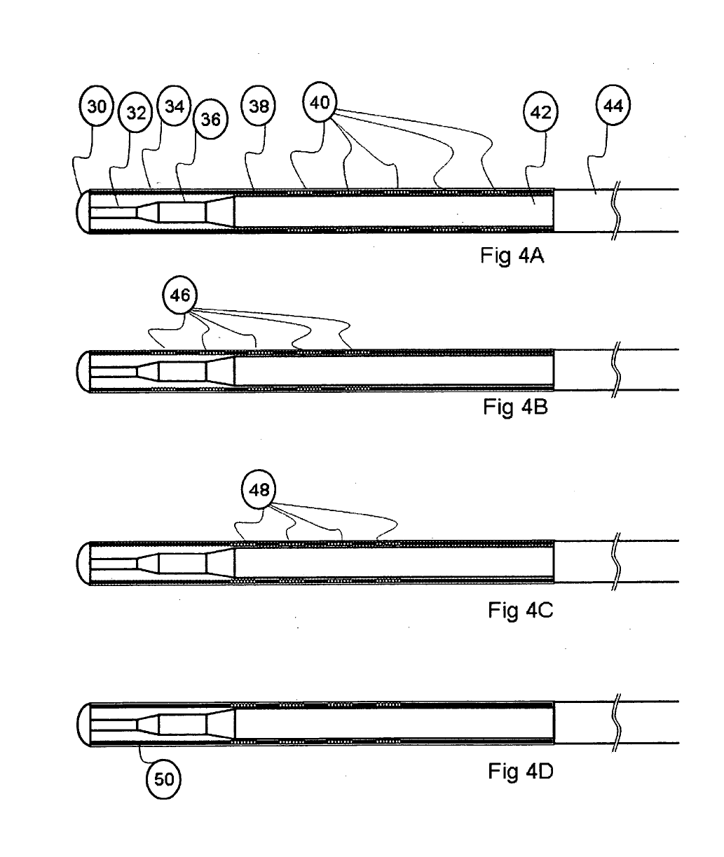

FIG. 4 show a preferred embodiment according to the invention. FIG. 4A shows a guidewire having similar sections/zones as a prior art guidewire, however the guidewire includes segmented coils 40 as a part of the overall coil cover of the guidewire 38. It can be seen that the guidewire is built in a typical structure: A distal tip 30, followed by a flexible zone core member 32, followed by a thicker core member 36 (called the “stent zone”), and finally a thicker core member 42, followed by the rest of the guidewire 44. The coils covering the different zones, are no longer unified and passive as in the prior art, but rather include several sections 40, that are electrically connected to each other, and can transmit electrical current, hence generating a magnetic flux.

FIG. 4A shows that the active coil sections 40 cover the core member 42, containing 5 separate members.

FIG. 4B shows a similar embodiment, however the active coil segments 46 are positioned more distally along the guidewire. The number of the separated segments in this figure is again 5.

FIG. 4C shows another typical embodiment, where the number of active coils sections 48, is 4.

FIG. 4D shows another typical embodiment, where the active coil sections are not embedded in the passive coil section, as shown in FIGS. 4A, 4B and 4C, but rather the gaps between the active coils are filled with other types of materials 50, preferably bio-compatible materials.

It is apparent to the man skilled in the art that both, the exact position of the active coils, as well as the number of active coils, may be varied.

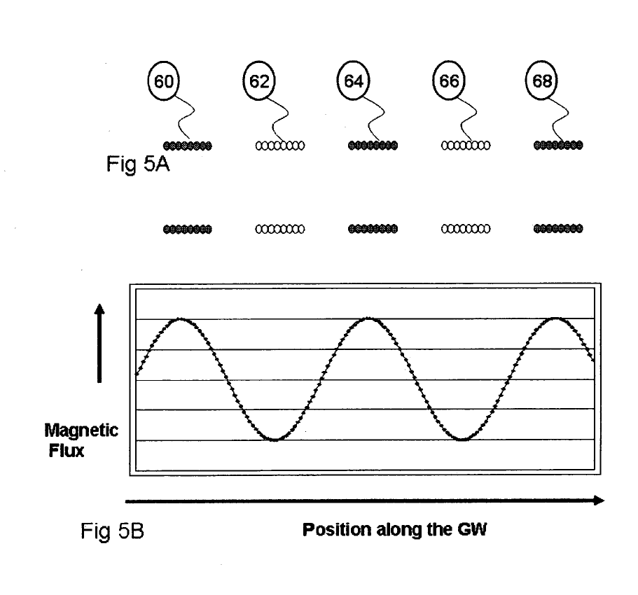

FIG. 5A shows a magnified cross section of 5 single layer coil segments. The coils are electrically connected in serial to each other in such a manner so that the direction of the current in coils 60, 64 and 68 is clockwise, while the current in coils 62 and 66 is counter clockwise. The resulting magnetic flux vs. the position along the guidewire is schematically shown in FIG. 5B. Other electrical connections between the coils are may be provided, resulting in different magnetic flux behavior. It is apparent to the man skilled in the art that the number of coils, number of layers for each coil, as well as their electrical connection may be varied.

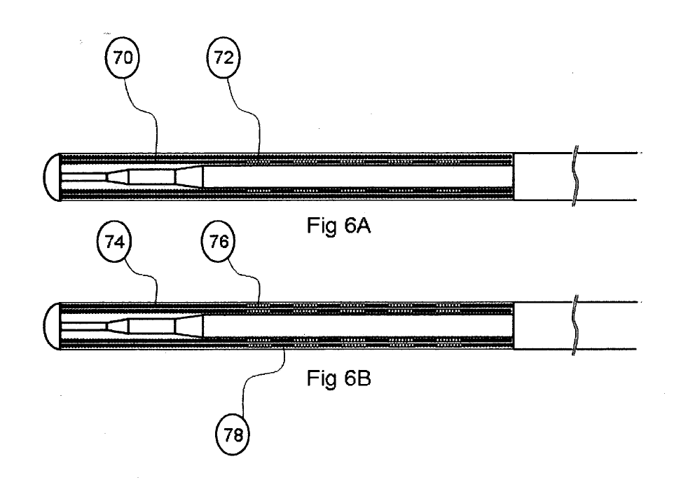

Such configuration may be adapted to the guidewire as shown in FIG. 4 and following FIG. 6.

Assuming such configuration is indeed adapted to the guidewire shown in FIG. 4, it will result in a variable magnetic field along the guidewire section enveloped by the active coils.

FIG. 6 shows two additional alternative embodiments for implementing the active coils into the guidewire. FIG. 6A shows a configuration where the active coils 72 are formed in a single layer, covered by a passive coil layer 70. FIG. 6B shows active coils segments 76 of two layer each, the gaps between active segments being filled with two layers of passive coils 78. Dual (or more then single) layers of active coils 76 result in higher (approximately double) magnetic flux generated by the coils, however it also results in s the guidewire to be thicker than if it had only a single layer.

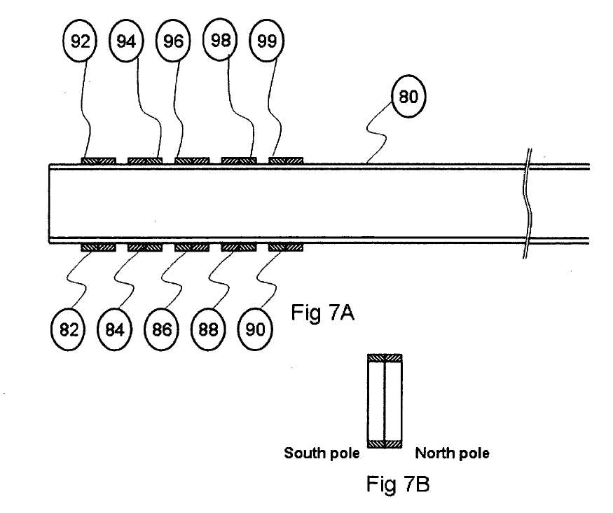

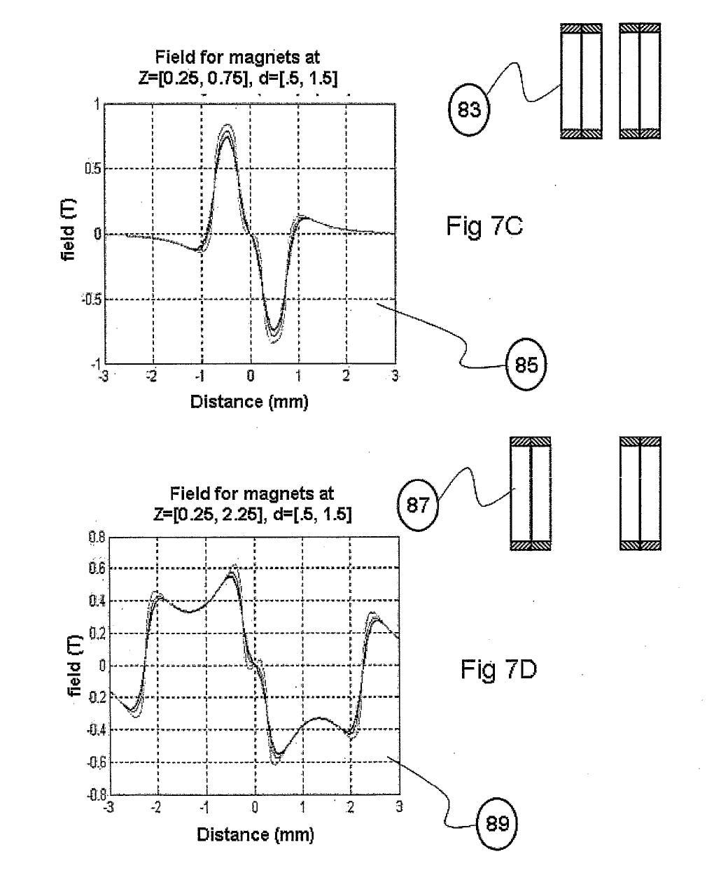

FIG. 7 show a preferred embodiment of a magnetic catheter. FIG. 7B shows a hollow cylinder shaped magnet, having a north and south magnetic poles on opposite facets of the cylinder. The magnet bid preferably is made of rare earth magnetic materials such as NdFb48. A series of such bids, are attached to a catheter as shown in FIG. 7A. In this figure five bids (82,84,86,88 and 90), are attached externally to catheter 80. In this preferred embodiment the direction of the bids is shown schematically in the figure: faces 92, 94, 96, and 99 are the south poles of the magnets, while the opposite faces of each magnet is the North Pole.

The dependency of the gradient of the magnetic field on the gap between the magnetic bids is shown in FIGS. 7C and 7D. FIG. 7C shows the an apparatus of 2 magnetic bids separated 0.5 mm from each other 83, the resulting magnetic filed is shown in graph 85.

FIG. 7D shows the magnetic bids 87, separated 2.0 mm from each other, the resulting magnetic field is shown in graph 89. These alternatives shown in FIGS. 7C and 7D, generates a relatively high magnetic field gradient, necessary to generate magnetic force.

However it should be emphasized that different number of magnetic bids, other orientation of the magnets as well as other dimensions of the gaps (either equally or not equally gapped) are covered by this invention.

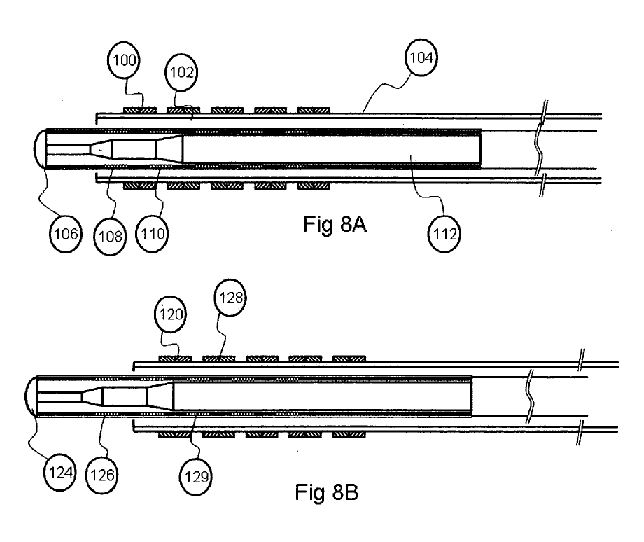

FIG. 8 shows a guidewire threaded via the catheter. FIG. 8A shows a specific optional positioning of the GW 112, inside the catheter 104. The maximum magnetic force will be developed once the magnet coil 110 is exactly in between two magnetic bids 100 and 102. The usage of several magnets, having a certain gap, and several coils having the same period as the magnets, will result in multiplication of the magnetic force accordingly. Other configurations of the magnet/coil arrangement may also be employed.

FIG. 8B shows a different positioning of the guidewire inside the catheter, where coil 129 is overlapped by magnet bids 128 and 120.

In both cases a magnetic force is developed on the active coils of the guidewire. Upon driving the coils with alternating current, the magnetic force is alternating, resulting in longitudinal vibrations of the GW and hence the guidewire tip 124.

Theoretically, if the system would be perfectly symmetrical (i.e. the guidewire is exactly in the middle of the catheter), then the magnetic force would be purely in the longitudinal direction. However, as the guidewire is free to move inside the lumen of the catheter, and in most cases the guidewire and the catheter are bent while inserted into human vessel, the configuration deviates from symmetrical, resulting in a magnetic force that also has lateral components.

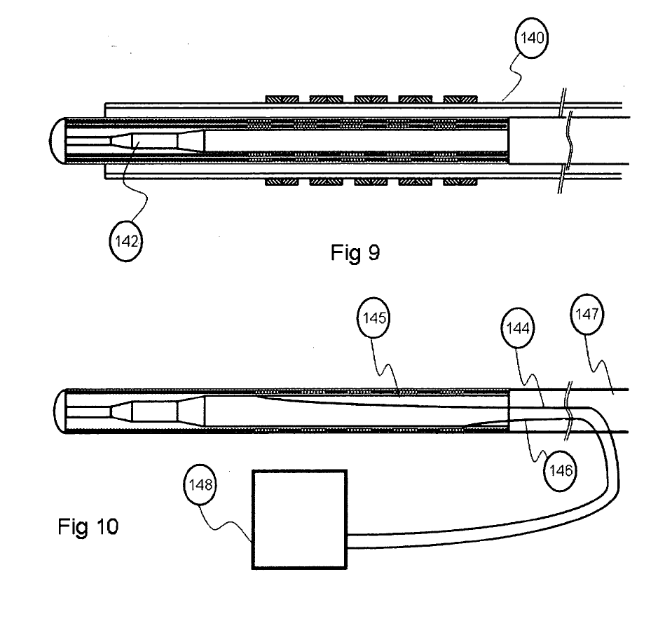

FIG. 9 shows another combination a guidewire and a catheter. In this embodiment a double layered guidewire 142 is threaded via a catheter 140. The principle of generating magnetic force in this apparatus is similar to that described in FIGS. 8A and 8B.

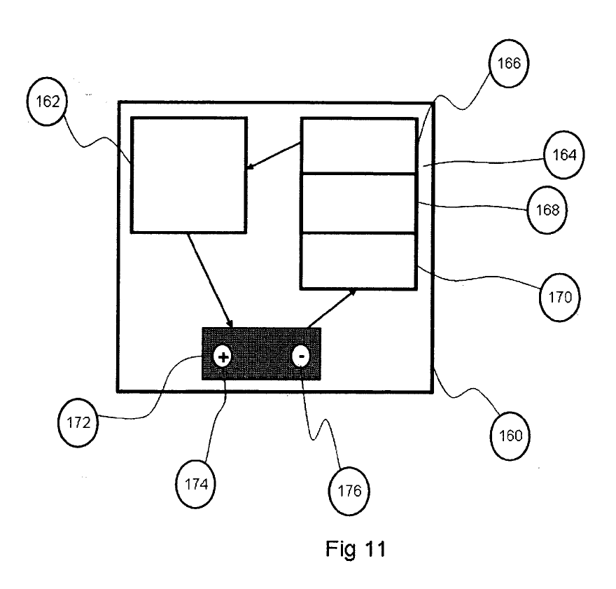

FIG. 10 shows the leads emerging from the active coils, and strung along the guidewire all the way to its proximal side. In this figure, the active coils are connected in serial to each other, where only two leads 144 and 146 are reach the proximal side of the guidewire. These two leads are then connected to an electrical current driver 148. When current is driven through the leads, a magnetic filed is generated by the coils and induces a magnetic force. There are several possibilities to string the leads from the coils to the proximal guidewire, one being externally to the guidewire itself. A second possibility is to make the guidewire itself hollow inside, enabling the wiring of the leads through this lumen. Alternatively, since in a typical guidewire the core member of the guidewire 147 that follows the coils section 145 of the guidewire, is made of conductive material, it can be split into two parallel core members, electrically isolated from each other, where one serves as the positive lead, while the second, one as the negative lead.

At the proximal side of the guidewire the leads (of any type) can either end as bare leads—directly connected to the external electrical current driver, or make use of a connector, preferable a miniature connector, for ease of operation.

It should be noted, that the number of the leads may also be greater than two, and depends on the number of the independent coil segments implemented in the proximal portion of the guidewire.

FIG. 11 shows a schematic configuration of the external current driver, and its derived capabilities.

The external driver 160, generally comprises the following modules:
A termination module 172, used to connect the leads of the guidewire by providing a positive and a negative signals, 174 and 176 accordingly.
A signal output generator 162: The current fed through these terminations to the guidewire is generated by a signal output generator 162. The figure illustrates a single signal generator, although multiple generators are may also be employed, connected to multiple termination modules. This generator can provide current into the guidewire coils, at different amplitudes, frequencies, and shapes. Preferably the output signal generator will provide sinus, rectangle, and triangle signals, at amplitude of up to 10 amperes, and frequencies at the range of 5 to 1000 Hz.
A signal analyzer comprising of three main sub-modules:
   Signal input module 170. Responsible for sensing the current fed into the coils, while measuring the voltage developing on said coils, at different frequencies.
   A signal analyzing module 168. Responsible to analyze the measured signals delivered by the signal input module 170. Such analysis is done by means of digital signal processing. More specifically the signals delivered from the signal input module 170, can be used to measure the following parameters:
      The resistance of the coils. Since the resistance of the coils depends in a known manner on the temperature that surrounds the coil, it is therefore possible to remotely measure and analyze the temperature of the coils, and upon reaching a pre-determined temperature limit, to automatically control the auto/manual control module 166 to stop output signal delivered by the signal output generator 162.
      The impedance of the coils, vs. the frequency of the signal fed into the coils, hence providing data about the organ that is in proximity of the GW tip that may be used for increasing the safety of the drilling feature of the guidewire. More particularly, usage of such continuous measuring of the impedance of the coils can be employed to determine whether the guidewire has dissected into the vessel wall, or may be even performing perforation to the vessel wall.
   An auto/manual control module 166. Which receive the analysis from the signal analyzing module 168, which enables for automatic and/or manual control of the signal output generator 162. In the manual control mode the operator/physician can control the driver in various ways, such as using a keyboard to enter controlled parameters, leg pedal to start/stop the driver, rotating knob etc. The automatic mode enables some or all of the manual functions to be performed automatically, and hence reduces the work load of the physician. For example, upon analyzing a too high temperature developing on the active coils, the driver automatically stops its operation, until temperature returns to normal/allowed level. It should be mentioned that although only a single signal output is shown in the drawing, multiple outputs enabling the simultaneous and independent driving of several coils leads may also be employed.

It should be mentioned that although only a single signal output is shown in the drawing, multiple outputs may be employed enabling the simultaneous and independent driving of several coil leads.

All of the above mentioned parameters are given by way of example only, and may be changed in accordance with the different requirements of the various embodiments of the present invention. Thus, the abovementioned parameters should not be construed as limiting the scope of the present invention in any way. In addition, it is to be appreciated that the different tubes, wires, magnets, and other members, described hereinabove may be constructed in different shapes (e.g. having oval, square etc. form in plan view) and sizes differing from those exemplified in the preceding description.

The above examples and description have been provided only for the purpose of illustration, and are not intended to limit the invention in any way. As will be appreciated by the skilled person, the invention can be carried out in a great variety of ways, employing more than one technique from those described above, all without exceeding the scope of the invention.

1. A wire shaped device for inducing in-vivo vibrations in a body passageway or an organ, comprising distal and proximal portions, wherein said distal portion being connected to said proximal portion, and wherein at least one coil is mounted over said distal portion, and wherein said at least one coil comprises at least one layer of windings capable of being electrically fed with electrical current by means of electrical leads connected to said at least one coil.

2. A device according to claim 1, wherein said coil is split into two or more segments, and wherein each of said segments is capable of being individually and independently fed with electrical current.

3. A device according to claim 2, wherein the coil is split into at least two segments, and wherein part or all of said segments are electrically connected to each other in a serial connection arranged such that the direction of the electrical currents through each segment is opposite to the direction of the electrical current passing through an adjacent segment.

4. A device according to claim 2, wherein the number of layers in each coil segment in the two or more coil segments is different, for all, or for portions, of said coil segments, and wherein two or more of said coil segments are arranged such that equal, or varying, gaps are provided therebetween, and wherein said gaps comprise passive coils, or any type of material capable of filling said gaps or portions thereof.

5. A device according to claim 4, wherein said filling material is a bio-compatible material and/or a thermally conductive material.

6. A device according to claim 1, wherein the at least one coil comprises a coating, wherein said coating is a hydrophilic coating, a Teflon based coating, a bio-compatible coating, or a thermally conducting coating.

7. A device according to claim 1, wherein the electrical leads of the at least one coil are provided at the proximal side of said device, and wherein said electrical leads are attached along the side of said device by an electrically insolating shrinkable-sleeve, or placed in an inner lumen provided in the proximal portion of said device.

8. A device according to claim 1, wherein a proximal portion of said device is made from an electrically conducting material, and wherein the electrical leads of the device are implemented by at least two electrically isolated sections of said electrically conducting material.

9. A device according to claim 1, wherein the electrical leads are terminated with bare leads, or by an electrical connector.

10. A device according to claim 2, wherein the device is a type of guidewire.

11. A device according to claim 1, wherein the at least one coil is made from a material selected from the following group of materials: copper, silver, platinum, gold.

12. A catheter tube comprising one or more magnetic elements attached along a length of said catheter tube, wherein said magnetic elements comprise a bio-compatible coating and wherein the shape of said magnetic elements is cylindrical, hollowed cylinder, box, hollowed box shape, partially cylindrically shaped, partially of hollowed cylindrical shape, partially box-shaped, or partially of hollowed box.

13. A catheter tube according to claim 12, wherein the one or more magnetic elements are made of rare earth magnetic materials.

14. A catheter tube according to claim 12, wherein the one or more magnetic elements are embedded into the wall of said catheter.

15. A catheter tube according to claim 12, wherein the one or more magnetic elements are attached to the outer surface of the catheter tube.

16. A catheter tube according to claim 12, wherein the one or more magnetic elements are attached to the inner surface of the catheter tube.

17. A catheter tube according to claim 12, wherein the one or more magnetic elements are arranged along said catheter tube such that gaps are obtained between adjacent magnetic elements.

18. A catheter tube according to claim 17, wherein the one or more magnetic elements are equally spaced from each other.

19. A catheter tube according to claim 17, wherein the one or more magnetic elements are unequally spaced from each other.

20. An apparatus for inducing in vivo vibrations in a body passage or organ, comprising:
a catheter tube comprising one or more lumens and means for generating a magnetic field capable of inducing a magnetic field within one or more sections of said one or more lumens; and
a guidewire contained in one of said lumens, wherein said guidewire comprises electrically controlled magnetic coupling means adapted to produce vibrations in said guidewire responsive to said electrically controlled magnetic coupling means.

21. The apparatus according to claim 20, wherein the guidewire comprises distal and proximal portions, wherein said distal portion being connected to said proximal portion, and wherein at least one coil is mounted over said distal portion, and wherein said at least one coil comprises at least one layer of windings capable of being electrically fed with electrical current by means of electrical leads connected to said at least one coil;
wherein each of said segments is capable of being individually and independently fed with electrical current; and
wherein the catheter tube comprises one or more magnetic elements attached along a length of said catheter tube, wherein said magnetic elements comprise a bio-compatible coating and wherein the shape of said magnetic elements is cylindrical, hollowed cylinder, box, hollowed box shape, partially cylindrically shaped, partially of hollowed cylindrical shape, partially box-shaped, or partially of hollowed box.

22. An apparatus according to claim 21, wherein the guidewire is electrically connected via its leads to an external driver and sensing unit.

23. An apparatus according to claim 22, wherein the external driver and sensing unit comprises:
means capable of generating at least one electrical current output and feeding the same into the leads of at least one coil of said guidewire;
means capable of detecting at least one voltage level developed over said guidewire leads and one current level delivered into said leads;
means capable of analyzing the voltage and current signals detected at different frequencies, and providing clinical data indications accordingly;
means capable of automatically controlling in close loop the current driver based on said analyzed clinical data; and
means capable of manually controlling the current provided via the driver output.

24. An apparatus according to claim 23, wherein the clinical data indications relate to the type of organ/material that is positioned in proximity of the guidewire.

25. An apparatus according to claim 23, wherein the clinical data indications relate to the temperature of the coils of the guidewire, and wherein the means capable of automatically controlling in close loop the current driver are adapted to automatically stop the current supplied by said current driver according to said clinical data indications whenever the temperature of said coils is beyond a predetermined level.
